(19) United States
(12) Patent Application Publication
(10) Pub. No.: US 2008/0166786 A1
(43) Pub. Date: Jul. 10, 2008

(54) Pump Unit, Syringe Unit, Method for Delivering Particles, and Method for Delivering Cells

(76) Inventor: Shusaku Nishiyama, Kawasaki (JP)

Correspondence Address: ARENT FOX LLP, 1050 CONNECTICUT AVENUE, N.W., SUITE 400, WASHINGTON, DC, 20036, US

(21) Appl. No.: 11/885,438

(22) PCT Filed: Mar. 10, 2005
(86) PCT No.: PCT/JP2005/004180

§ 371 Date: Aug. 31, 2007

(51) Int. Cl. C12N 1/00; C12M 1/00; F04B 17/04; F04B 23/02

(52) U.S. Cl. 435/243; 435/307.1; 417/476; 417/413.2

(57) ABSTRACT

The present invention relates to a pump unit or the like which feeds particles in a liquid with particles dispersed therein to a predetermined area. An object of the present invention is to obtain a sufficient solution feeding resolution while inhibiting mixture of bubbles. The pump unit includes a moving mechanism 13 that moves a pump 12 and a reservoir 11 relative to each other to vary the positional relationship between a tip 1221 of the pump 12 and an edge 112 of the opening 111 between a separating relationship in which the tip 1221 is separated upward from the edge 112 of the opening 111 in a liquid reserved in the reservoir 11 and a pressing relationship in which the tip 1221 is pressed against the edge 112. In the separating positional relationship, the pump 12 performs a sucking operation to take particles C into its interior. In the pressing relationship, the pump 12 performs an ejecting operation to deliver the particles C. The moving mechanism 13 varies the positional relationship between the separating relationship and the pressing relationship with the tip 1221 immersed in the liquid reserved in the reservoir 11.

### TECHNICAL FIELD

The present invention relates to a pump unit that feeds particles in a liquid with the particles dispersed therein to a predetermined area, a method for delivering particles which is carried out using the pump unit, a syringe unit that feeds cells in a suspension with the cells dispersed therein to a microchannel, and a method for delivering cells which is carried out using the syringe unit.

### BACKGROUND ART

Various apparatuses have been proposed which serve as systems that deliver particles mixed and dispersed in a liquid (see, for example, Documents 1 and 2). Here, by way of example, description will be given of a syringe unit used in an apparatus that introduces a substance into blood-derived cells or stem cells in the field of medicine (see, for example, Patent Document 3). Patent Document 3 shows the substance introducing apparatus for massive continuous processing. A syringe unit installed in the substance introducing apparatus uses a syringe to feed cells into a microchannel.

In general, to introduce a substance into blood-derived cells or stem cells, blood-derived cells or stem cells to be introduced are first extracted from a living organism and subjected to a dispersing treatment using trypsin or the like. The cells are then dispersed in a culture medium. Then, the cells dispersed and suspended in the culture medium are fed into a microchannel via a syringe. The cells are allowed to flow to a predetermined treatment position via the culture medium. The cells reaching the treatment position are captured by a sucking mechanism. A pouring system introduces an agent or the like into any site of each cell.

In the syringe unit described in Patent Document 3, the tip of the syringe internally filled with the culture medium is connected to the microchannel via a tube. The syringe then performs an ejecting operation to feed cells into the microchannel through the tube. Here, when bubbles are mixed in the microchannel filled with the culture medium, the mixed bubbles hinder the delivery of the cells to make the solution feeding through the microchannel unstable, even with the ejecting operation of the syringe. Various factors contribute to mixing the bubbles into the microchannel. The major factor is air entering the tube during syringe replacement when the syringe having ejected the culture medium is replaced with a syringe filled with a culture medium.

At the treatment position, to allow the sucking mechanism to reliably capture the cells, the migration of the cells is monitored on the basis of image analysis via a CCD camera. Monitoring the migration of the cells requires a resolution sufficient for a very small amount of liquid fed. Blood-derived cells have a diameter of about 5 to 20 μm in a suspended condition. Delivery and capture of cells are facilitated provided that the cross section of the microchannel has the minimum size required to contain the cells. Thus, when the cross section of the microchannel is shaped like a substantial square of 50 μm on a side, the cells need to be delivered at 500 μm/sec in order to obtain a sufficient solution feeding resolution. This requires a flow rate of 1.25 nL/sec. Thus, a syringe with a small inner diameter may be used to obtain a sufficient solution feeding resolution.

Patent Document 1: Japanese Utility Model Laid-Open No. 5-13198

Patent Document 2: Japanese Patent Laid-Open No. 2001-258545

Patent Document 3: Japanese Patent Laid-Open No. 2004-166653

### DISCLOSURE OF THE INVENTION

### Problem to be Solved by the Invention

However, the use of a syringe with a small inner diameter reduces the amount of liquid ejected during a single operation. The syringe unit described in Patent Document 3 thus requires frequent syringe replacements, resulting in the mixture of a large amount of bubble into the microchannel.

In view of these circumstances, an object of the present invention is to provide a pump unit and a syringe unit which can obtain a sufficient solution feeding resolution while inhibiting the mixture of bubbles, as well as a method for delivering particles which is carried out using the pump unit and a method for delivering cells which is carried out using the syringe unit.

### Solution for Solving the Problem

To accomplish the above object, the present invention provides a pump unit that feeds particles in a liquid with the particles dispersed therein to a predetermined area, the pump unit including:

a reservoir that reserves the liquid and that has an opening at a bottom of the reservoir, the opening connected to the predetermined area;

a pump that performs a sucking operation of sucking the liquid through a tip thereof and an ejecting operation of ejecting the sucked liquid from the tip; and

a moving mechanism that moves the pump and the reservoir relative to each other to vary a positional relationship between the tip of the pump and an edge of the opening, between a separating relationship in which the tip of the pump is separated upward from the edge of the opening with the tip staying in the liquid reserved in the reservoir and a pressing relationship in which the tip is pressed against the edge of the opening,

wherein in the separating positional relationship, the pump performs the sucking operation to bring the particles into the pump, and in the pressing positional relationship, the pump performs the ejecting operation to deliver the particles, and

the moving mechanism varies the positional relationship between the separating relationship and the pressing relationship with the tip immersed in the liquid reserved in the reservoir.

With the pump unit in accordance with the present invention, while the positional relationship is varying between the separating relationship and the pressing relationship, the pump tip is not drawn up from the liquid level of the liquid. Further, the need for syringe replacement is eliminated to prevent the entry of air. This inhibits the mixture of bubbles. Further, even when a syringe with a small inner diameter is used to obtain a sufficient solution feeding resolution, the repetition of an ejecting operation and a sucking operation does not result in the disadvantageous mixture of bubbles because the need for syringe replacement is eliminated to prevent the entry of air. Therefore, the pump unit in accordance with the present invention can provide a sufficient solution feeding resolution while inhibiting the mixture of bubbles.

Further, in the pump unit in accordance with the present invention, the pump can preferably be separated from the pump unit.

This allows the pump to be replaced with a new one after continuous feeding. Furthermore, a maintenance operation such as cleaning of the pump is easy.

Here, the moving mechanism may include urging means that urges the tip of the pump toward the edge of the opening and a cam mechanism that separates the tip of the pump from the edge of the opening against an urging force of the urging means. Alternatively, the moving mechanism may be a piezo actuator.

Further, the pump unit in accordance with the present invention preferably includes removal means that removes particles or bubbles present between the tip of the pump and the edge of the opening in the separating positional relationship.

The removal means may blow a fluid against the tip of the pump.

The removal means can prevent the particles from being sandwiched between the tip of the pump and the edge of the opening when the positional relationship changes from the separating relationship to the pressing relationship. Further, air dissolved into the liquid reserved in the reservoir may appear as bubbles. Removal of the thus appearing bubbles enables the possible mixture of bubbles to be reliably inhibited.

In a preferred aspect of the pump unit in accordance with the present invention, the pump starts the ejecting operation while the positional relationship is being changed from the separating relationship to the pressing relationship by the moving mechanism, to remove particles or bubbles present between the tip and the edge of the opening, or

the moving mechanism moves the pump and the reservoir relative to each other in a horizontal direction in the separating positional relationship, and

the reservoir has a brush member that has upward extending bristles implanted at a bottom thereof so that relative movement of the pump and the reservoir in a horizontal direction causes the brush member to slidably rub against the tip of the pump to remove attachments from the tip.

These aspects can reliably inhibit the sandwiching of particles and the mixture of the bubbles.

Moreover, in the pump unit in accordance with the present invention, the edge of the opening in the reservoir preferably projects upward from a part of the bottom which surrounds the edge. Further, the edge of the opening more preferably has a projecting tip surface that is an upward projecting curved surface.

The projecting edge reduces the area of a part of the edge which is contacted by the pump tip, increasing the contact pressure of the pump tip. This also reduces the possibility of sandwiching the particles between the pump tip and the edge of the opening. The possibility is further reduced when the projecting amount is larger than the diameter of the particle. Moreover, forming the edge into an upward projecting curved surface allows the particles to roll down the projecting tip surface, further reducing the possibility of sandwiching the particles between the pump tip and the edge of the opening.

Furthermore, in the pump unit in accordance with the present invention, the pump preferably repeats the sucking operation and the ejecting operation in the separating positional relationship to disperse particles unevenly distributed in the reservoir.

Long, continuous operation is likely to result in the uneven distribution of the particles, for example, precipitation of the particles at the bottom. With the above arrangement, the entry and exit of the liquid into and from the pump tip stirs the interior of the reservoir to disperse the unevenly distributed particles.

In another preferred aspect, the pump unit in accordance with the present invention includes:

supply means that supplies the liquid to the reservoir;

monitor means that monitors the liquid level of the liquid reserved in the reservoir; and

a control section that, when the monitor means indicates that the liquid level is lower than a predetermined height, causes the supply section to supply the liquid to the reservoir.

According to this aspect, even if long, continuous operation lowers the liquid level of the reservoir, the pump tip is not located above the liquid level. This prevents the possible mixture of bubbles in spite of long, continuous operation.

To accomplish the above object, the present invention provides a syringe unit that feeds cells in a suspension with the cells dispersed therein into a microchannel, the syringe unit including:

a reservoir that reserves the suspension and that has an opening at a bottom of the reservoir, the opening connected to the microchannel;

a syringe that performs a sucking operation of sucking the suspension through a tip thereof and an ejecting operation of ejecting the sucked suspension from the tip; and

a moving mechanism that moves the syringe and the reservoir relative to each other to vary a positional relationship between the tip of the syringe and an edge of the opening, between a separating relationship in which the tip of the syringe is separated upward from the edge of the opening with the tip staying in the suspension reserved in the reservoir and a pressing relationship in which the tip is pressed against the edge of the opening, and

wherein in the separating positional relationship, the syringe performs the sucking operation to bring the cells into the syringe, and in the pressing positional relationship, the syringe performs the ejecting operation to deliver the cells, and

the moving mechanism varies the positional relationship between the separating relationship and the pressing relationship with the tip immersed in the suspension reserved in the reservoir.

To accomplish the above object, the present invention provides a method for delivering particles, the method including:

a first step of reserving a liquid with the particles dispersed therein in a reservoir having an opening at a bottom thereof, the opening connected to a predetermined area;

a second step of, while a positional relationship between an edge of the opening and a tip of a pump that performs a sucking operation of sucking the liquid into an interior through a tip thereof and an ejecting operation of ejecting the sucked liquid from the tip toward an exterior is a separating relationship in which the tip of the pump is separated upward from the edge of the opening with the tip staying in the liquid reserved in the reservoir, causing the pump to perform the sucking operation to take the particles into the pump;

a third step of changing the positional relationship from the separating relationship to a pressing relationship in which the tip of the pump is pressed against the edge of the opening, with the tip of the pump immersed in the liquid reserved in the reservoir;

a fourth step of, in the pressing positional relationship, causing the pump to perform the ejecting operation to carry out the second step to deliver the particles taken into the pump; and

a fifth step of changing the positional relationship from the pressing relationship to the separating relationship with the tip of the pump immersed in the liquid reserved in the reservoir,

wherein performing the second to fifth steps is repeated.

The method for delivering particles according to the present invention prevents the pump tip from being drawn up from the liquid level of the liquid. Further, syringe replacement is not carried out, preventing the entry of air. This inhibits the mixture of bubbles. Further, even when a syringe with a small inner diameter is used to obtain a sufficient solution feeding resolution, the repetition of the second to fifth steps does not result in the disadvantageous mixture of bubbles because the need for syringe replacement is eliminated to prevent the entry of air. Therefore, the method for delivering particles in accordance with the present invention can provide a sufficient solution feeding resolution while inhibiting the mixture of bubbles.

To accomplish the above object, the present invention provides a method for delivering cells, the method including:

a first step of reserving a suspension with the cells dispersed therein in a reservoir having an opening at a bottom thereof, the opening connected to a microchannel area;

a second step of, while a positional relationship between an edge of the opening and a tip of a syringe that performs a sucking operation of sucking the suspension into an interior through a tip thereof and an ejecting operation of ejecting the sucked suspension from the tip toward an exterior is a separating relationship in which the tip of the syringe is separated upward from the edge of the opening with the tip staying in the suspension reserved in the reservoir, causing the syringe to perform the sucking operation to take the cells into the syringe;

a third step of changing the positional relationship from the separating relationship to a pressing relationship in which the tip of the syringe is pressed against the edge of the opening, with the tip of the syringe immersed in the suspension reserved in the reservoir;

a fourth step of, in the pressing positional relationship, causing the syringe to perform the ejecting operation to carry out the second step to deliver the cells taken into the syringe; and

a fifth step of changing the positional relationship from the pressing relationship to the separating relationship with the tip of the syringe immersed in the suspension reserved in the reservoir,

wherein performing the second to fifth steps is repeated.

### EFFECT OF THE INVENTION

According to the present invention, the present invention provides a pump unit and a syringe unit which can obtain a sufficient solution feeding resolution while inhibiting the mixture of bubbles, as well as a method for delivering particles which is carried out using the pump unit and a method for delivering cells which is carried out using the syringe unit.

### BRIEF DESCRIPTION OF THE DRAWINGS

FIG. 1 is a perspective view showing a substance introducing apparatus with a syringe unit in accordance with a first embodiment installed therein.

FIG. 2 is a diagram showing that the syringe shown in FIG. 1 is performing a sucking operation.

FIG. 3 is a diagram showing that the syringe shown in FIG. 1 is performing an ejecting operation.

FIG. 4 is a diagram showing an example in which a moving mechanism shown in FIGS. 2 and 3 and provided in the syringe unit shown in FIG. 1 has been replaced with a different one.

FIG. 5 is a diagram showing that removal means provided in the syringe unit in accordance with the present embodiment is removing cells and bubbles.

FIG. 6 is a diagram showing that a brush member provided in place of the removal means shown in FIG. 5 is removing bubbles or cells.

FIG. 7 is a flowchart showing a procedure of introducing a substance into cells using the substance introducing apparatus shown in FIG. 1.

FIG. 8 is a diagram showing that cells or bubbles are being removed by a syringe.

FIG. 9 is a diagram showing that an edge of an opening in a reserving well is formed higher to prevent cells from being sandwiched.

FIG. 10 is a diagram showing that an area that surrounds the edge of the opening in the reserving well is formed lower to prevent cells from being sandwiched.

FIG. 11 is a diagram showing an example in which the edge of the opening in the reserving well shown in FIG. 9 is formed into a curved surface.

FIG. 12 is a diagram showing that cells precipitated at the bottom of the reserving well is being dispersed by the syringe.

FIG. 13 is a diagram showing a syringe unit for which step S12 shown in FIG. 7 is automated.

### BEST MODE FOR CARRYING OUT THE INVENTION

Embodiments of the present invention will be described with reference to the drawings.

First, description will be given of a syringe unit that is an embodiment of a pump unit in accordance with the present invention.

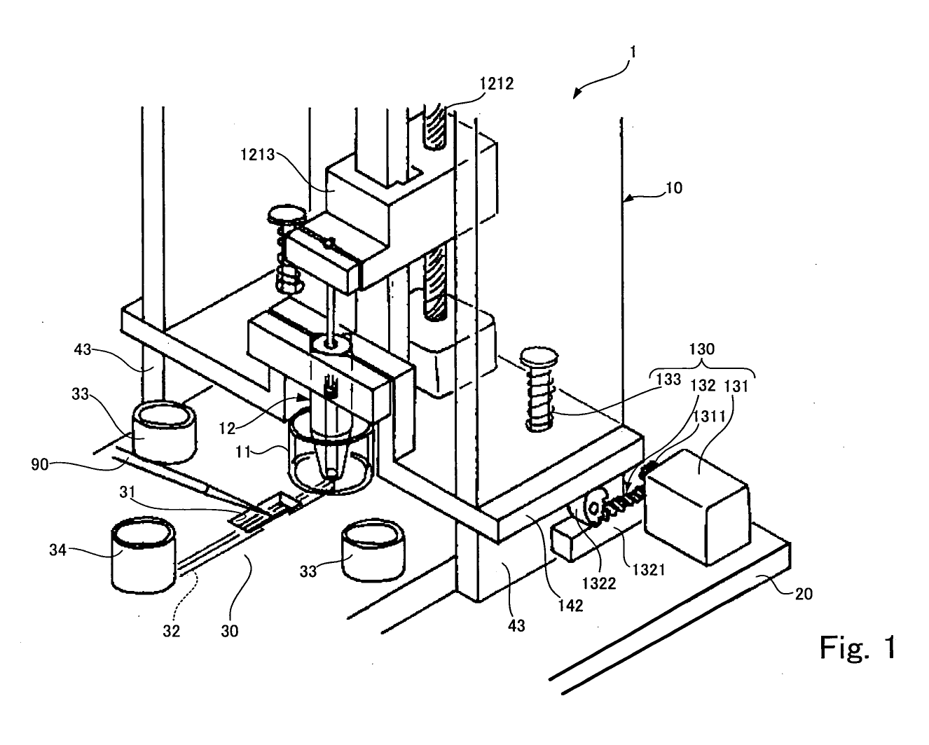

FIG. 1 is a perspective view showing a substance introducing apparatus having a syringe unit in accordance with a first embodiment installed therein.

The substance introducing apparatus 1 shown in FIG. 1 is used in the medical field to introduce agents or the like into blood-derived cells or stem cells. The substance introducing apparatus 1 has a syringe unit 10, a base 20, and a channel plate 30 which also correspond to an embodiment of a syringe unit in accordance with the present invention. The channel plate 30 is installed on the base 20 and has a treatment window 31 in a front surface thereof. A microchannel 32 is formed in the channel plate 30 so as to extend through the treatment window 31. The syringe unit 10 feeds cells into the microchannel 32, with the fed cells migrating through the microchannel 32. A sucking mechanism (not shown) is installed on a back surface of the channel plate 30 at the position where the treatment window 31 is formed (treatment position). Cells migrating through the microchannel 32 are captured by the sucking mechanism. At the treatment position, to allow the sucking mechanism to reliably capture the cells, the migration of the cells is monitored on the basis of image analysis via a CCD camera. FIG. 1 shows that an agent or the like is being introduced into the captured cells (not shown) via a capillary 90. Culture medium wells 33 in which a culture medium (in which cells are not dispersed) is reserved are provided on the respective sides of the treatment position in the channel plate 30. The culture medium wells 33 are connected to the microchannel 32 inside the channel plate 30. The culture medium in the culture medium wells 33 forms an interfacial flow along an inner wall of the microchannel 32 under a Venturi effect to assist delivery of the cells. Moreover, a treated cell well 34 is provided downstream of the microchannel 32 to store treated cells into which the agent or the like has been introduced.

The syringe unit 10 is installed upstream of the microchannel 32. The syringe unit 10 includes a reserving well 11, a syringe 12, and a moving mechanism 13. Further, the syringe 12 includes a linear moving mechanism that moves linearly in a vertical direction to perform a sucking operation and an ejecting operation.

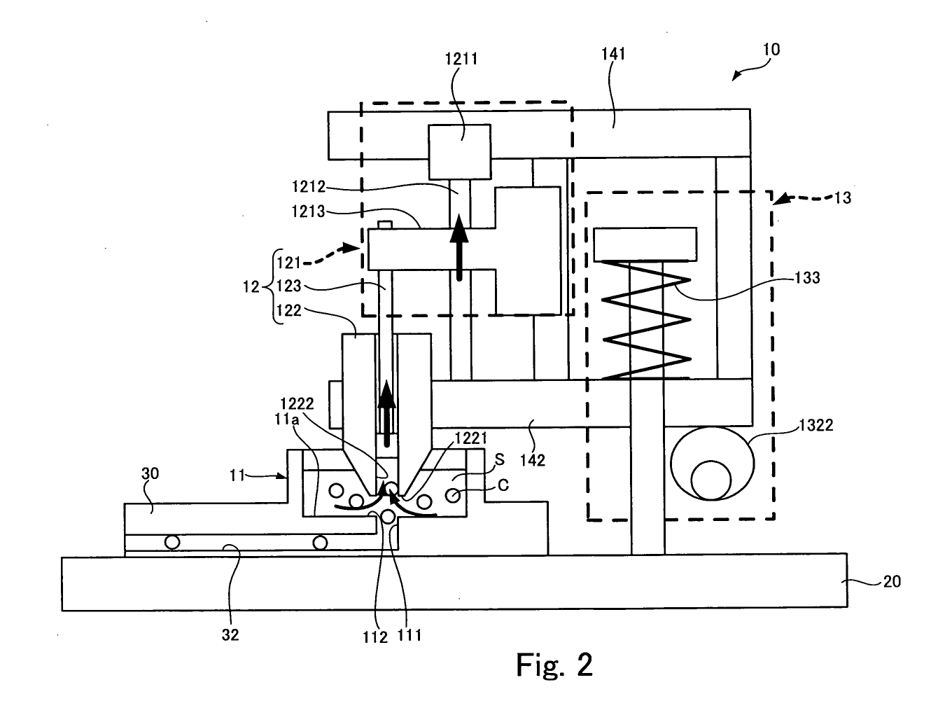

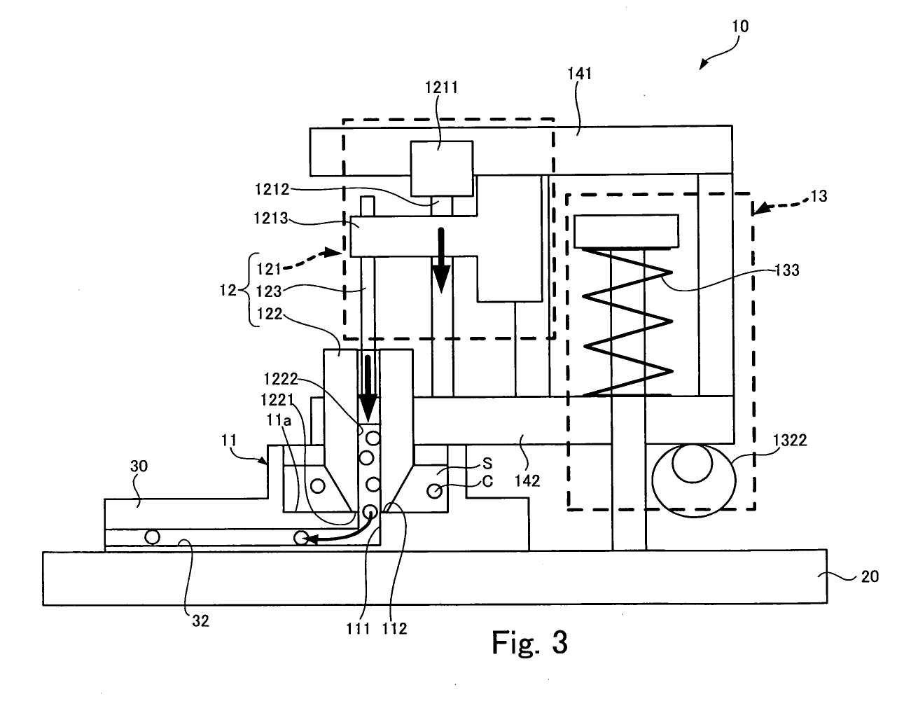

Now, the syringe unit 10 will be described in detail with reference to FIGS. 2 and 3 in addition to FIG. 1.

FIG. 2 is a diagram showing that the syringe shown in FIG. 1 is performing a sucking operation. FIG. 3 is a diagram showing that the syringe shown in FIG. 1 is performing an ejecting operation.

The reserving well 11 is provided in an area of the channel plate 30 which is located upstream of the microchannel 32. An opening 111 connecting to the microchannel 32 is formed at a bottom 11a of the reserving well 11. A suspension S with cells C dispersed therein is reserved in the reserving well 11.

The syringe 12 has a syringe barrel 122 and a syringe plunger 123 in addition to the linear moving mechanism 121. As shown in FIG. 2, the linear moving mechanism 121 has a motor 1211 fixed to an upper frame 141 of the syringe unit 10, a ball screw 1212 extending in the vertical direction to transmit rotation of the motor 1211, and a guide member 1213 penetrated by the ball screw 1212. The guide member 1213 is moved up and down along the ball screw 1212 by forward and backward rotation of the motor 1211. A rear end of the syringe plunger 123 is releasably attached to a tip of the guide member 1213. The syringe barrel 122 is releasably attached to a lower frame 142 of the syringe unit 10. Accordingly, rotation of the motor 1211 in a predetermined direction raises the syringe plunger 123 to suck the suspension S reserved in the reserving well 11 into an interior 1222 of the syringe barrel 122 through a tip 1221 of the syringe barrel 122 as shown in FIG. 2 (sucking operation). FIG. 2 shows that a sucking operation is being performed to take the cells C into the interior 1222 of the syringe barrel 122. In contrast, the motor 1211 rotates backward to push down the syringe plunger 123 to eject the sucked suspension S from the tip 1221 as shown in FIG. 3 (ejecting operation). FIG. 3 shows that an ejecting operation is being performed to deliver the cells C taken into the interior 1222 of the syringe barrel 122, to the microchannel 32 via the opening 111.

The syringe unit 10 in accordance with the present embodiment can consecutively perform a sucking operation and an ejecting operation. It is unnecessary to replace the syringe plunger 123 and syringe barrel 122 with new ones during a continuous process. However, if the syringe plunger 123 and syringe barrel 122 need to be replaced with new ones after the continuous process has been finished, the replacement can be easily carried out because both the syringe plunger 123 and syringe barrel 122 are releasably attached. Further, the syringe can be removed for maintenance such as a treatment for sterilizing the syringe. This offers improved operability.

Moreover, the syringe barrel 122 has a small inner diameter (for example, 0.5 to 1.0 mm) to provide a sufficient solution feeding resolution.

The moving mechanisms 13 are provided on the respective sides of the lower frame 142. However, FIG. 1 shows only one of the moving mechanisms 13. Each of the moving mechanisms 13 has a cam motor 131, an eccentric cam member 132, and a spring member 133. Further, as shown in FIG. 1, paired height defining blocks 43 are installed on the base 20 on the respective sides of the channel plate 30. FIG. 1 shows the lower frame 142 of the syringe unit 10 is placed on the paired height defining blocks 43. The spring member 133 urges the syringe unit 10 downward until the lower frame 142 of the syringe unit 10 is placed on the paired height defining blocks 43. That is, the spring member 133 is urging means for urging the tip 1221 of the syringe barrel 122 toward an edge 112 of the opening 111 in the reserving well 11 (see FIGS. 2 and 3). In the syringe unit 10 shown in FIG. 3, the lower frame 142 is placed on the paired height defining blocks 43, with the tip 1221 of the syringe barrel 122 pressed against the edge 112 of the opening 111 in the reserving well 11. Accordingly, the positional relationship between the tip 1221 of the syringe barrel 122 and the edge 112 of the opening 111 in the reserving well 11, shown in FIG. 3, is the pressing relationship in which the tip 1221 of the syringe barrel 122 is pressed against the edge 112 of the opening 111 in the reserving well 11.

As shown in FIG. 1, the cam motor 131 is fixed on the base 20 and has a pinion gear 1311 secured to its rotating shaft. The eccentric cam member 132 is composed of a rack member 1321 and an eccentric cam 1322. The rack member 1321 slides on the base 20 in conjunction with rotation of the cam motor 131. The eccentric cam 1322 is rotatably supported by the paired height defining blocks 43 and rotates in conjunction with sliding of the rack member 1321. The eccentric cam 1322 rotates so that its cam surface pushes up the lower frame 142 of the syringe unit 10 placed on the paired height defining blocks 43. That is, the eccentric cam member 132 separates the tip 1221 of the syringe barrel 122 from the edge 112 of the opening 111 in the reserving well 11, in the suspension S against the urging force of the spring member 133. The positional relationship between the tip 1221 of the syringe barrel 122 and the edge 112 of the opening 111 in the reserving well 11, shown in FIG. 2, is the separating relationship in which the tip 1221 of the syringe barrel 122 is separated upward from the edge 112 of the opening 111, in the suspension S reserved in the reserving well 11. Thus, the moving mechanism 13 moves the syringe 12 up and down to vary the positional relationship between the tip 1221 of the syringe barrel 122 and the edge 112 of the opening 111 in the reserving well 11 between the separating relationship shown in FIG. 2 and the pressing relationship shown in FIG. 3. Moreover, the moving mechanism 13 in accordance with the present embodiment varies the positional relationship between the separating relationship and the pressing relationship with the tip 1221 of the syringe barrel 122 immersed in the suspension S reserved in the reserving well 11. Consequently, with the syringe unit 10 in accordance with the present embodiment, while the positional relationship is being varied between the separating relationship and the pressing relationship, the tip 1221 of the syringe barrel 122 is not drawn up from the liquid level of the suspension S. Thus, syringe replacement during continuous treatment is not required, preventing the entry of air. This inhibits bubbles from mixing into the microchannel 32. Further, the small inner diameter of the syringe barrel 122 provides a sufficient solution feeding resolution while inhibiting the mixture of bubbles.

Now, description will be given of a variation of the moving mechanism 13 shown in FIGS. 2 and 3. In the description below, components with the same names as those of the components described above are denoted by the same reference numerals.

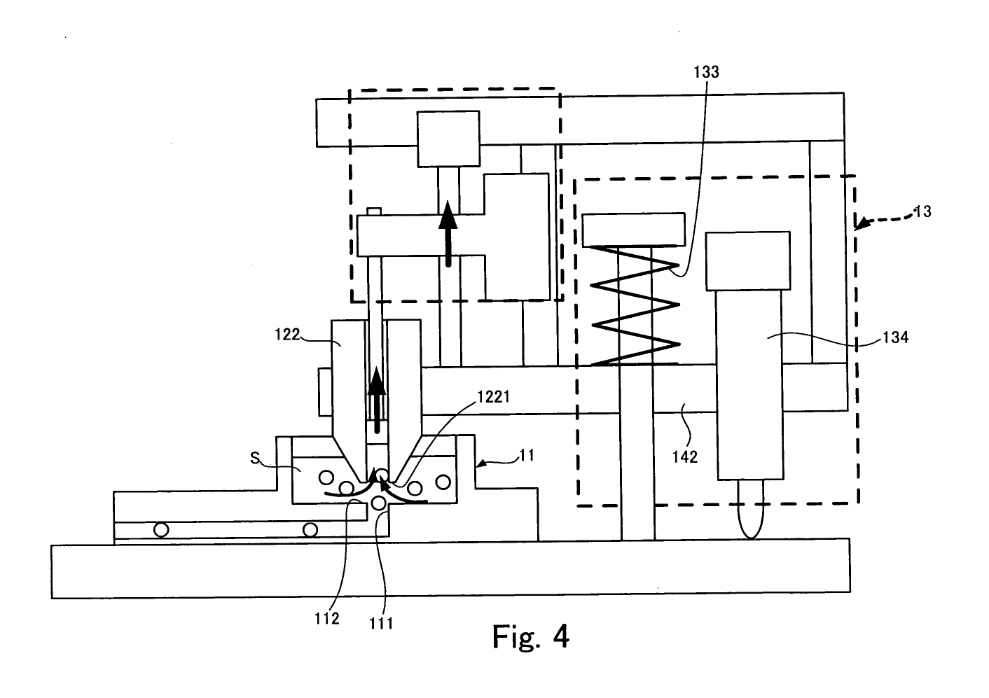

FIG. 4 is a diagram showing an example in which the moving mechanism shown in FIGS. 2 and 3 and provided in the syringe unit shown in FIG. 1 has been changed to a different one.

The moving mechanism 13 shown in FIG. 4 also has the spring member 133 as urging means, but has a piezo actuator 134 in place of the two components, the cam motor 131 and eccentric cam member 132. The piezo actuator 134 is fixed to the lower frame 142 on the base 20. The piezo actuator 134 utilizes a piezoelectric effect or an inverse piezoelectric effect to extend to separate the tip 1221 of the syringe barrel 122 from the edge 112 of the opening 111 in the reserving well 11, in the suspension S against the urging force of the spring member 133. FIG. 4 shows the extended piezo actuator 134 as well as the separating positional relationship.

Both moving mechanisms described above moves the syringe 12 up and down to vary the positional relationship between the separating relationship and the pressing relationship. However, the positional relationship may be varied between the separating relationship and the pressing relationship by moving the reserving well 11 up and down. That is, the moving mechanism has only to vary the positional relationship between the separating relationship and the pressing relationship by moving the syringe 12 and the reserving well 11 relative to each other.

Moreover, the syringe unit 10 in the present embodiment has removal means for removing, in the separating positional relationship, cells and bubbles present between the tip 1221 of the syringe barrel 122 and the edge 112 of the opening 111 in the reserving well 11.

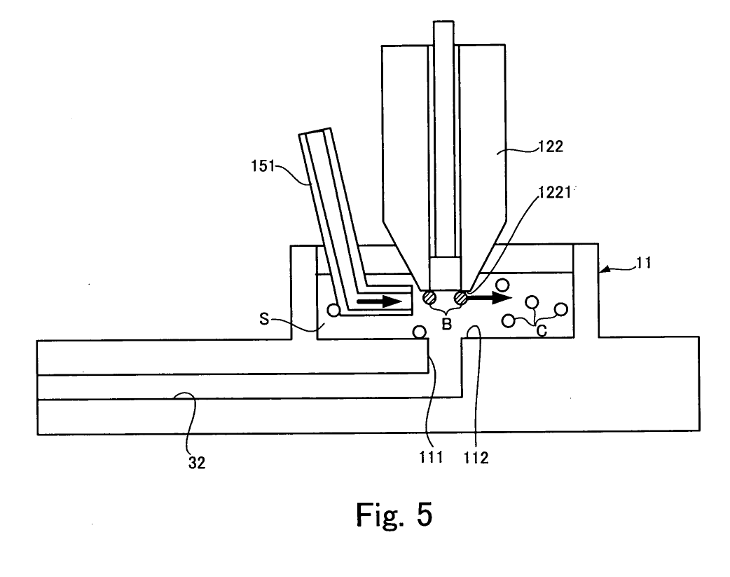

FIG. 5 is a diagram showing that the removal means provided in the syringe unit in accordance with the present embodiment is removing cells and bubbles.

FIG. 5 shows that bubbles B are attached to the tip 1221 of the syringe barrel 122. First time the tip of the syringe barrel 122 is immersed in the suspension S reserved in the reserving well 11, the bubbles B may be attached to the tip 1221 of the syringe barrel 122. Further, air dissolved in the suspension reserved in the reserving well 11 may appear as bubbles. Moreover, when the positional relationship changes from the separating relationship to the pressing relationship, cells C present between the tip 1221 of the syringe barrel 122 and the edge 112 of the opening 111 may be sandwiched between the tip 1221 and the edge 112. Removal means 151 shown in FIG. 5 sprays a culture medium between the tip 1221 of the syringe barrel 122 and the edge 112 of the opening 111 in the reserving well 11. The culture medium sprayed by the removal means 15 removes the bubbles B and cells C present between the tip 1221 and the edge 112. Consequently, the syringe unit 10 in accordance with the present embodiment makes it possible to prevent the cells C from being sandwiched between the tip 1221 and the edge 112, while reliably inhibiting the bubbles B from entering the microchannel 32.

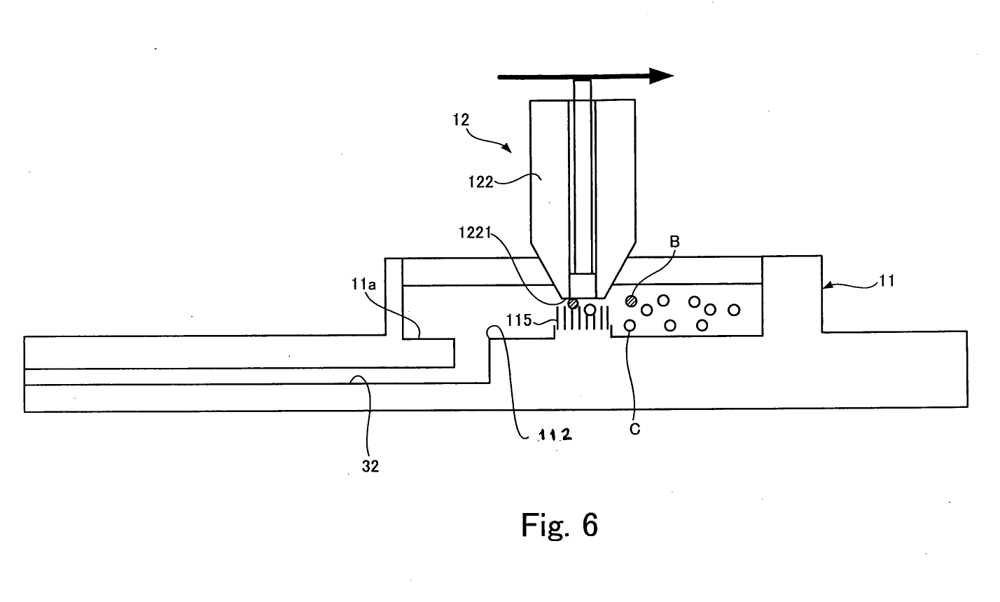

FIG. 6 shows that in place of the removal means shown in FIG. 5, a brush member is provided to remove bubbles and cells.

The moving mechanism 13 in this case can also move the syringe 12 in the horizontal direction (see an arrow in FIG. 6) in the separating positional relationship. The moving mechanism 13 has only to move the syringe 12 and the reserving well 11 relative to each other in the horizontal direction. Further, the reserving well 11, shown in FIG. 6, has a brush member 115 at a bottom 11a. The brush member 115 is implanted at the bottom 11a so as to extend upward. When the syringe 12 moves in the horizontal direction, the brush member 115 slidably rubs against the tip 1221 of the syringe barrel 122 to remove the bubbles B and cells C attached to the tip 1221. This also makes it possible to prevent the cells C from being sandwiched between the tip 1221 and the edge 112, while reliably inhibiting the bubbles B from entering the microchannel 32.

Subsequently, description will be given of a procedure of introducing an agent or the like into cells using the substance introducing apparatus 1, shown in FIG. 1. This procedure includes the procedure of a method for delivering cells in accordance with an embodiment of a method for delivering particles in accordance with the present invention.

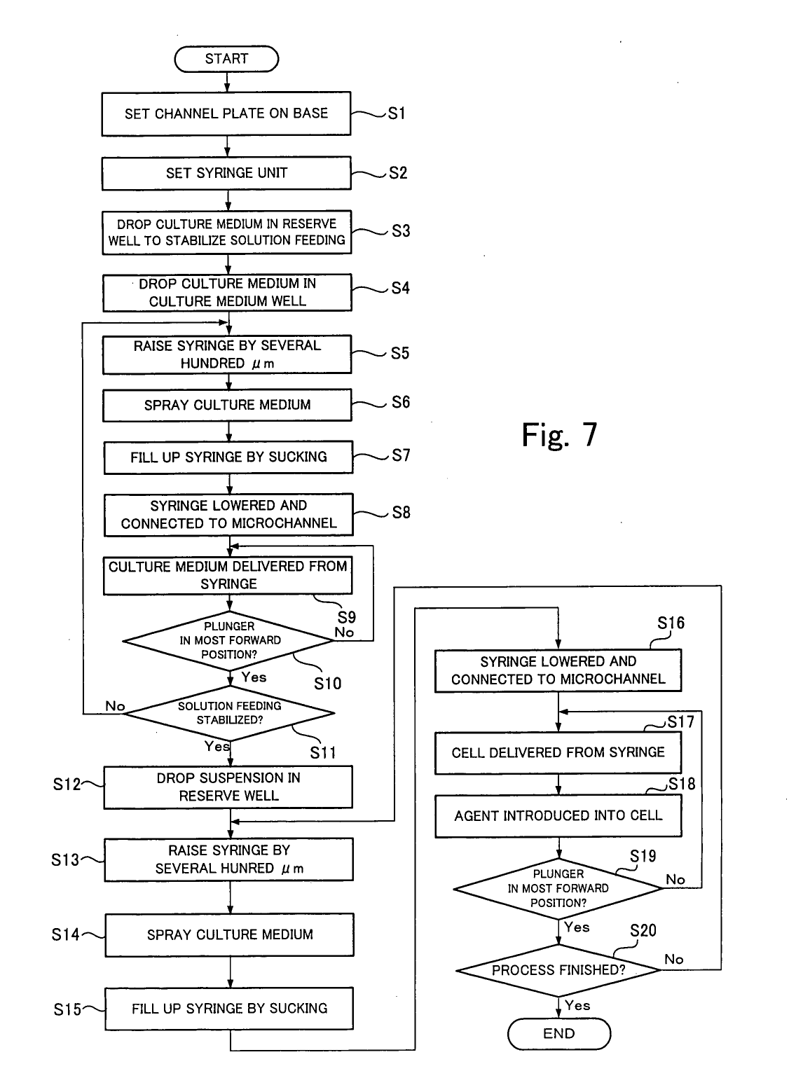

FIG. 7 is a flowchart showing a procedure of introducing a substance into cells using the substance introducing apparatus, shown in FIG. 1.

In the flowchart shown in FIG. 7, a substance such as an agent is introduced after the solution feeding state in the microchannel 32 has been stabilized. First, the channel plate 20 is set on the base 20, shown in FIG. 1 (step S1). Then, the syringe unit 10 is set (step S2). With the syringe unit 10 set, the urging force of the spring member 133 establishes the pressing positional relationship shown in FIG. 3. Subsequently, a culture medium with cells not dispersed therein is dropped into the reserving well 11 in order to stabilize solution feeding (step S3). The culture medium is also dropped into the culture medium well 33 (step S4). Then, the cam motor 131 of the moving mechanism 13 is rotated to allow the cam surface of the eccentric cam 1322 to push down the lower frame 142 to raise the syringe 12 by several hundred μm (for example, 200 to 300 μm) (step S5). This changes the positional relationship from the pressing relationship to the separating relationship, shown in FIG. 2. Then, with the separating relationship maintained, the removal means 151, shown in FIG. 5, sprays the culture medium between the tip 1221 of the syringe barrel 122 and the edge 112 of the opening 111 in the reserving well 11 (step S6) to remove the bubbles B present between the tip 1221 and the edge 112. Subsequently, in the separating relationship, the syringe 12 is caused to perform a sucking operation to fill the culture medium into the interior 1222 of the syringe barrel 122 (step S7). Then, the cam motor 131 is rotated to cancel the pushup operation by the cam surface of the eccentric cam 1322 so that the urging force of the spring member 133 returns the positional relationship to the pressing relationship, shown in FIG. 3 (step S8). That is, the syringe 12 is lowered to connect the tip 1222 of the syringe barrel 122 to the opening 111. Subsequently, in the pressing state, the syringe 12 is caused to perform an ejecting operation to carry out step S7. The culture medium filled in the interior is thus delivered to the microchannel 32 via the opening 111 (step S9). The process then determines whether or not the plunger 123 is in its most forward position (step S10). That is, the process determines whether or not the syringe plunger 123 has been completely pushed down to finish the ejecting operation. If the ejecting operation has not been finished, it is continued (step S9). If the ejecting operation has been finished, the process proceeds to step S11. In step S11, the process determines whether or not the solution feeding state of the microchannel 32 has been stabilized. If the solution feeding state is unstable, the process proceeds to step S5. If the solution feeding state is stable, the process proceeds to step S12 to start a substance introducing process.

In the substance introducing process, first, in step S12, a suspension with the cells C dispersed therein is dropped into the reserving well 11 (this corresponds to an example of a first step in accordance with the present invention). Then, as in the case of step S5, the syringe 12 is raised by several hundred μm (step S13) to change the positional relationship to the separating relationship with the tip 1221 of the syringe barrel 122 immersed in the culture medium reserved in the reserving well 11. Then, with the separating relationship maintained, the culture medium is sprayed as in the case of step S6 (step S14). The process then proceeds to step S15. In step S14, cells and bubbles resulting from air dissolved in the suspension reserved in the reserving well 11 are removed from between the tip 1221 of the syringe barrel 122 and the edge 112 of the opening 111. In step S15, in the separating relationship, the syringe 12 is caused to perform a sucking operation to take the cells C into the interior 1222 of the syringe barrel 122 (this corresponds to an example of a second step in accordance with the present invention). In step S16 following step S15, as in the case of step S8, the positional relationship is returned to the pressing relationship, shown in FIG. 3, with the tip 1221 of the syringe barrel 122 immersed in the suspension reserved in the reserving well 11 (this corresponds to an example of a third step in accordance with the present invention). The process then proceeds to step S17. In step S17, in the pressing relationship, the syringe 12 is caused to perform an ejecting operation to carry out step S15 to deliver the cells C filled in the interior to the microchannel 32 via the opening 111 (this corresponds to an example of a fourth step in accordance with the present invention). Then, at the treatment position, where the treatment window 31 is formed as shown in FIG. 1, the cells are captured and a substance such as an agent is introduced into the cells (step S18). Then, as in the case of step S10, the process determines whether or not the syringe plunger 123 is in its most forward position (step S19). If the syringe plunger 123 has not reached the most forward position, the ejecting operation is continued (step S17). If the syringe plunger 123 has reached the most forward position, the process proceeds to step S20. In step S20, the process determines whether or not the substance has been introduced into a required number of cells, that is, whether or not the substance introducing process has been finished. If the substance introducing process has not been finished, the process returns to step S13 to repeat steps S13 to S20 until the substance introducing process is finished. On the other hand, once the substance introducing process is finished, the flowchart ends.

In the procedure of the introduction of a substance into cells described above, the tip 1221 of the syringe barrel 122 is not drawn up from the liquid level of the suspension during steps S5 to S20. Thus, the need for syringe replacement is eliminated to prevent the entry of air. This inhibits bubbles from mixing into the microchannel 32. Further, even when the syringe has a small inner diameter to obtain a sufficient solution feeding resolution, the repetition of steps S13 to S20 does not result in the disadvantageous mixture of bubbles because the need for syringe replacement is eliminated to prevent the entry of air.

Now, description will be given of an applied example of the syringe unit in accordance with the present embodiment.

In the syringe unit 10 in accordance with the present embodiment, in step S6 or S14, shown in FIG. 7, the removal means 15, shown in FIG. 5, is used to spray the culture medium to remove the cells C and bubbles B. First, description will be given of an applied example in which the cells C and the bubbles B are removed during step SS16.

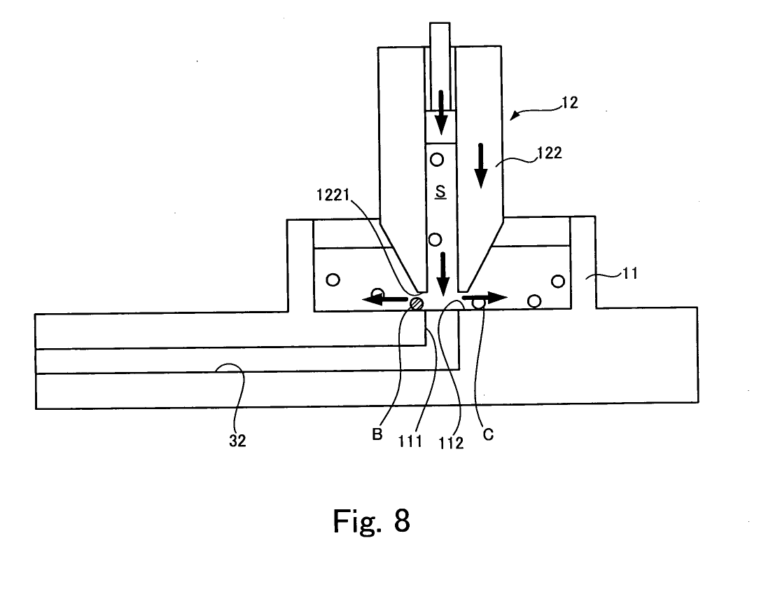

FIG. 8 is a diagram showing that cells and bubbles are being removed using the syringe.

The syringe 12 shown in FIG. 8 starts an ejecting operation while the positional relationship is changing from the separating relationship to the pressing relationship (step S16, shown in FIG. 7, is being carried out). The cells C and bubbles B present between the tip 1221 of the syringe barrel 122 and the edge 112 of the opening 111 are swept away toward a peripheral wall of the reserving well 11 by the flow of the suspension S ejected from the tip 1221.

This more reliably inhibits the sandwiching of the cells C and the mixture of the bubbles B into the microchannel 32.

The sandwiching of the cells can also be prevented by projecting the edge of the opening in the reserving well upward from an area of the bottom surrounding the edge.

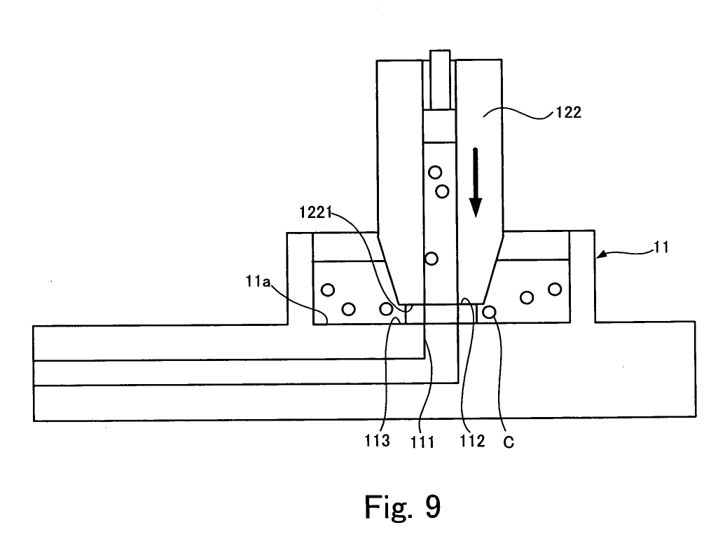

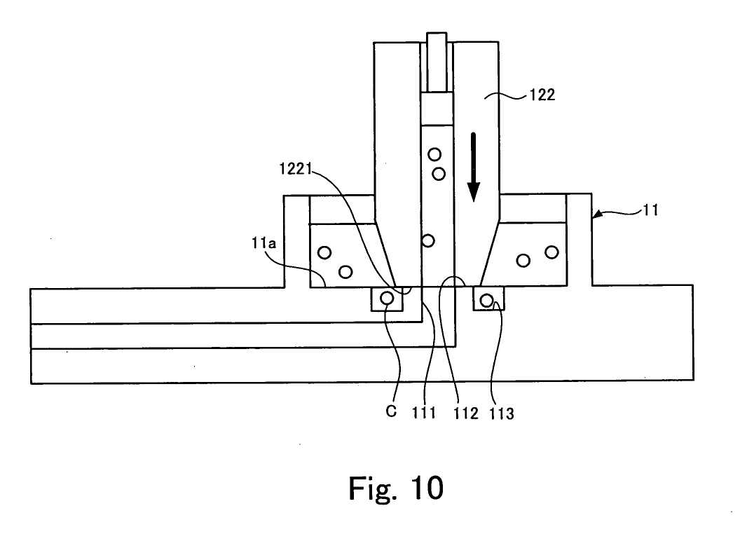

FIG. 9 is a diagram showing that the edge of the opening in the reserving well is formed higher to prevent cells from being sandwiched. FIG. 10 is a diagram showing that the edge of the opening in the reserving well is formed lower to prevent cells from being sandwiched.

The edge 112 of the opening 111 in the reserving well 11 shown in FIG. 9 projects from an area 113 surrounding the edge 112 of the bottom 11a, by at least a distance equal to the diameter of the cell C (5 to 20 μm in a floating state). Further, the area 113 of the bottom 11a shown in FIG. 10 which surrounds the edge 112 of the opening 111 is a groove recessed from the edge 112 by at least the distance equal to the diameter of the cell C. This reduces the possibility of sandwiching the cells between the tip 1221 of the syringe barrel 122 and the edge 112 of the opening 111. Further, the area 112 contacted by the tip 1221 of the syringe barrel 122 is reduced to increase the contact pressure of the tip 1221.

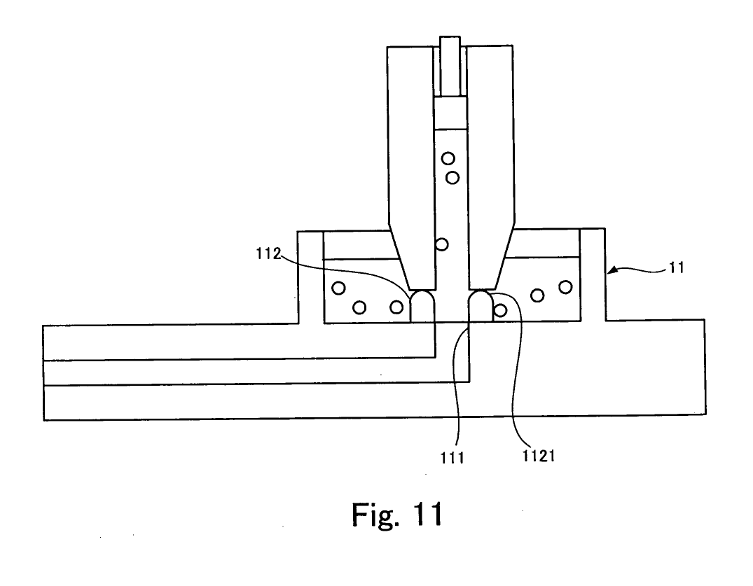

FIG. 11 is a diagram showing an example in which the edge of the opening in the reserving well shown in FIG. 9 is formed into a curved surface.

The edge 112 of the opening 111 in the reserving well 11 shown in FIG. 11 projects upward. A projecting tip surface 1121 forms an upward projecting curved surface. The upward projecting curved surface allows the cells C to roll down without remaining at the edge 112 of the opening 111. This further reduces the possibility of sandwiching the cells C.

During a long, continuous process, the uneven distribution of the cells C, for example, the precipitation of the cells C at the bottom 11a, is likely to occur in the reserving well 11. Thus, description will be given of an applied example in which the unevenly distributed cells C are dispersed.

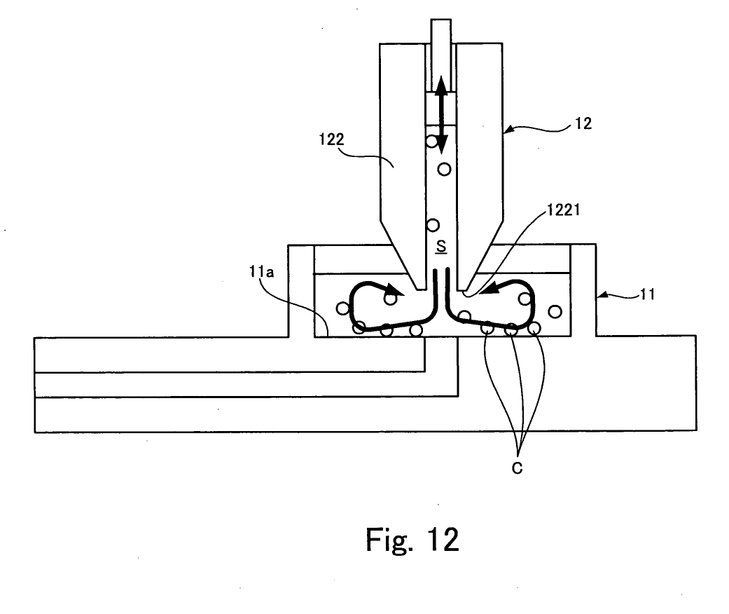

FIG. 12 is a diagram showing that cells precipitated at the bottom of the reserving well are being dispersed using the syringe.

The syringe 12 shown in FIG. 12 repeats a sucking operation and an ejecting operation in the separating positional relationship to force the suspension S into and out of the syringe barrel 122 through the tip 1221. The entry and exit of the suspension S stirs the interior of the reserving well 11 to disperse the cells C precipitated at the bottom 11a.

Finally, description will be given of the syringe unit 10 in which step S12, shown in FIG. 7, is automated to facilitate a long, continuous process.

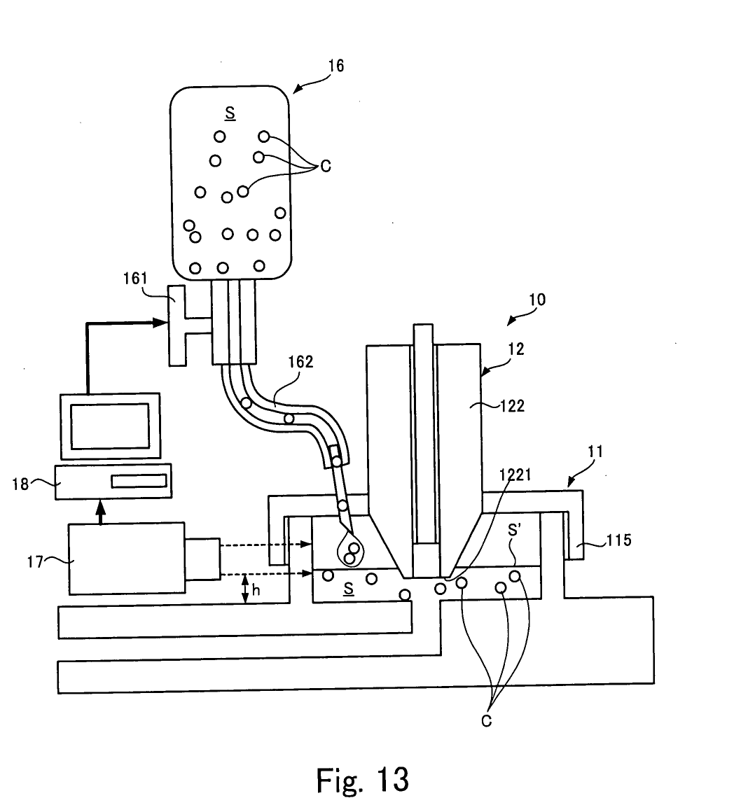

FIG. 13 is a diagram showing the syringe unit in which step S12, shown in FIG. 7, is automated.

The reserving well 11 provided in the syringe unit 10 shown in FIG. 13 has a cover 115 to prevent the entry of impurities. The syringe unit 10 has supply means 16, monitor means 17, and control section 18, in addition to the reserving well 11, syringe 12, and others. The supply means 16 supplies the reserving well 11 with the suspension S with the cells C dispersed therein. The supply means 16, shown in FIG. 13, has a valve 161 and a supply pipe 162. Opening the valve 161 feeds the suspension S to the reserving well 11 through the supply pipe 162. The monitor means 17 is a level sensor that monitors the liquid level S′ of the suspension S reserved in the reserving well 11. Further, on the basis of monitoring results from the monitor means 17, the control section 18 opens the valve 161 to supply the suspension S to the reserving well 11 if the liquid level S′ is lower than a predetermined height h. FIG. 13 shows the separating positional relationship, and the predetermined height h as used herein refers to a value somewhat larger than the height of the tip 1221 of the syringe barrel 122 in the separating relationship. The syringe unit 10 shown in FIG. 13 can prevent the tip 1221 of the syringe barrel 122 from lying above the liquid level S′ of the reserving well 11 even if the liquid level S′ lowers during a long, continuous process. Thus, even during a long, continuous operation, bubbles are inhibited from mixing into the microchannel 32.

As described above, the syringe unit 10 in accordance with the present embodiment can provide a sufficient solution feeding resolution while inhibiting bubbles from mixing into the microchannel 32. The present invention is not limited to the delivery of cells in the medical field but is applicable to various fields.

1. A pump unit that feeds particles in a liquid with the particles dispersed therein to a predetermined area, the pump unit comprising:
a reservoir that reserves the liquid and that has an opening at a bottom of the reservoir, the opening connected to the predetermined area;
a pump that performs a sucking operation of sucking the liquid through a tip thereof and an ejecting operation of ejecting the sucked liquid from the tip; and
a moving mechanism that moves the pump and the reservoir relative to each other to vary a positional relationship between the tip of the pump and an edge of the opening, between a separating relationship in which the tip of the pump is separated upward from the edge of the opening with the tip staying in the liquid reserved in the reservoir and a pressing relationship in which the tip is pressed against the edge of the opening,
wherein in the separating positional relationship, the pump performs the sucking operation to bring the particles into the pump, and in the pressing positional relationship, the pump performs the ejecting operation to deliver the particles, and
the moving mechanism varies the positional relationship between the separating relationship and the pressing relationship with the tip immersed in the liquid reserved in the reservoir.

2. The pump unit according to claim 1, wherein the pump can be separated from the pump unit.

3. The pump unit according to claim 1, wherein the moving mechanism comprises urging means that urges the tip of the pump toward the edge of the opening and a cam mechanism that separates the tip of the pump from the edge of the opening against an urging force of the urging means.

4. The pump unit according to claim 1, wherein the moving mechanism is a piezo actuator.

5. The pump unit according to claim 1, further comprising removal means that removes particles or bubbles present between the tip of the pump and the edge of the opening in the separating positional relationship.

6. The pump unit according to claim 5, wherein the removal means sprays a fluid between the tip of the pump and the edge of the opening.

7. The pump unit according to claim 1, wherein the pump starts the ejecting operation while the positional relationship is being changed from the separating relationship to the pressing relationship by the moving mechanism, to remove particles or bubbles present between the tip and the edge of the opening.

8. The pump unit according to claim 1, wherein the moving mechanism moves the pump and the reservoir relative to each other in a horizontal direction in the separating positional relationship, and
the reservoir has a brush member that has upward extending bristles implanted at a bottom thereof so that relative movement of the pump and reservoir in the horizontal direction causes the brush member to slidably rubs against the tip of the pump to remove attachments from the tip.

9. The pump unit according to claim 1, wherein the edge of the opening in the reservoir projects upward from a part of the bottom which surrounds the edge.

10. The pump unit according to claim 9, wherein the edge of the opening has a projecting tip surface that is an upward projecting curved surface.

11. The pump unit according to claim 1, wherein the pump repeats the sucking operation and the ejecting operation in the separating positional relationship to disperse particles unevenly distributed in the reservoir.

12. The pump unit according to claim 1, further comprising:
supply means that supplies the liquid to the reservoir;
monitor means that monitors the liquid level of the liquid reserved in the reservoir; and
a control section that, when the monitor means indicates that the liquid level is lower than a predetermined height, causes the supply section to supply the liquid to the reservoir.

13. A syringe unit that feeds cells in a suspension with the cells dispersed therein into a microchannel, the syringe unit comprising:
a reservoir that reserves the suspension and that has an opening at a bottom of the reservoir, the opening connected to the microchannel;
a syringe that performs a sucking operation of sucking the suspension through a tip thereof and an ejecting operation of ejecting the sucked suspension from the tip; and
a moving mechanism that moves the syringe and the reservoir relative to each other to vary a positional relationship between the tip of the syringe and an edge of the opening, between a separating relationship in which the tip of the syringe is separated upward from the edge of the opening with the tip staying in the suspension reserved in the reservoir and a pressing relationship in which the tip is pressed against the edge of the opening, and
wherein in the separating positional relationship, the syringe performs the sucking operation to bring the cells into the syringe, and in the pressing positional relationship, the syringe performs the ejecting operation to deliver the cells, and
the moving mechanism varies the positional relationship between the separating relationship and the pressing relationship with the tip immersed in the suspension reserved in the reservoir.

14. A method for delivering particles, the method comprising:
a first step of reserving a liquid with the particles dispersed therein in a reservoir having an opening at a bottom thereof, the opening connected to a predetermined area;
a second step of, while a positional relationship between an edge of the opening and a tip of a pump that performs a sucking operation of sucking the liquid into an interior through a tip thereof and an ejecting operation of ejecting the sucked liquid from the tip toward an exterior is a separating relationship in which the tip of the pump is separated upward from the edge of the opening with the tip staying in the liquid reserved in the reservoir, causing the pump to perform the sucking operation to take the particles into the pump;
a third step of changing the positional relationship from the separating relationship to a pressing relationship in which the tip of the pump is pressed against the edge of the opening, with the tip of the pump immersed in the liquid reserved in the reservoir;
a fourth step of, in the pressing positional relationship, causing the pump to perform the ejecting operation to carry out the second step to deliver the particles taken into the pump; and
a fifth step of changing the positional relationship from the pressing relationship to the separating relationship with the tip of the pump immersed in the liquid reserved in the reservoir,
wherein performing the second to fifth steps is repeated.

15. A method for delivering cells, the method comprising:
a first step of reserving a suspension with the cells dispersed therein in a reservoir having an opening at a bottom thereof, the opening connected to a microchannel;
a second step of, while a positional relationship between an edge of the opening and a tip of a syringe that performs a sucking operation of sucking the suspension into an interior through a tip thereof and an ejecting operation of ejecting the sucked suspension from the tip toward an exterior is a separating relationship in which the tip of the syringe is separated upward from the edge of the opening with the tip staying in the suspension reserved in the reservoir, causing the syringe to perform the sucking operation to take the cells into the syringe;
a third step of changing the positional relationship from the separating relationship to a pressing relationship in which the tip of the syringe is pressed against the edge of the opening, with the tip of the syringe immersed in the suspension reserved in the reservoir;
a fourth step of, in the pressing positional relationship, causing the syringe to perform the ejecting operation to carry out the second step to deliver the cells taken into the syringe; and
a fifth step of changing the positional relationship from the pressing relationship to the separating relationship with the tip of the syringe immersed in the suspension reserved in the reservoir,
wherein performing the second to fifth steps is repeated.
